(19) United States
(12) Patent Application Publication
(10) Pub. No.: US 2010/0189721 A1
(43) Pub. Date: Jul. 29, 2010

(54) ANTIBODY FORMULATIONS

(75) Inventors: Charlene E. Brisbane, King of Prussia, PA (US); Amol Sharad Ketkar, King of Prussia, PA (US)

Correspondence Address: GlaxoSmithKline;GLOBAL PATENTS -US, UW2220, P. O. BOX 1539, KING OF PRUSSIA, PA, 19406-0939, US

(73) Assignee: SMITHKLINE BEECHAM CORPORATION

(21) Appl. No.: 12/667,899

(22) PCT Filed: Jul. 3, 2008
(86) PCT No.: PCT/US08/69123

§ 371 Date: Jan. 6, 2010

Related U.S. Application Data
(60) Provisional application No. 60/948,218, filed on Jul. 6, 2007.

(51) Int. Cl. A61K 39/395; A61K 38/00

(52) U.S. Cl. 424/141.1; 514/12; 424/130.1

(57) ABSTRACT

This invention relates to a shear and temperature stable antibody formulations that are more stable than compared to a standard formulation (such as 30 mM citrate, 100 mM NaCl, pH 6.5). The present invention's shear and temperature stable antibody formulations show reduced precipitation when subjected to stress conditions but the standard formulation had aggregated. This result was unpredictable because thermodynamically the two formulations are similar as seen by their DSC (differential scanning calorimeter) profiles.

### FIELD OF THE INVENTION

This invention relates to a shear and temperature stable antibody formulations.

### BACKGROUND OF THE INVENTION

Proteins are larger and more complex than traditional organic and inorganic drugs (i.e. possessing multiple functional groups in addition to complex three-dimensional structures), and the formulation of such proteins poses special problems. For a protein to remain biologically active, a formulation must preserve the intact conformational integrity of at least a core sequence of the protein's amino acids while at the same time protecting the protein's multiple functional groups from degradation. Degradation pathways for proteins can involve chemical instability (i.e. any process which involves modification of the protein by bond formation of cleavage resulting in a new chemical entity) or physical instability (i.e. changes in the higher order structure of the protein). Chemical instability can result from deamidation, racemization, hydrolysis, oxidation, beta elimination or disulfide exchange. Physical instability can result from denaturation, aggregation, precipitation or adsorption, for example. The three most common protein degradation pathways are protein aggregation, deamidation and oxidation. Cleland et al. Critical Reviews in Therapeutic Drug Carrier Systems 10(4): 307-377 (1993).

There is a need for formulating a shear and temperature stable pharmaceutical formulation comprising a protein which is suitable for therapeutic use. In one embodiment the protein can be an antibody. In another embodiment the protein can be an IgG antibody. In yet another embodiment the protein can be an IgG1 antibody. In another embodiment the protein can be a monoclonal antibody. In another embodiment the protein can be an anti-Oncostatin M (anti-OSM) antibody, including but not limited to anti-OSM antibodies disclosed and described by SEQ ID NO: 35 for heavy chain and SEQ ID NO: 38 for light chain in WO2005/095457. In another embodiment the protein can be an anti-Myelin-associated glycoprotein (anti-MAG) antibody, including but not limited to anti-MAG antibodies disclosed and described by SEQ ID NO: 30 for heavy chain with disabled IgG1 constant region and SEQ ID NO: 31 for light chain in WO2004/014953. In another embodiment the antibody can be an anti-CD20 antibody, including but not limited to ofatumumab, rituximab, tositumomab, ocrelizumab (2H7.v16), 11B8 or 7D8 (disclosed in WO2004/035607), an anti-CD20 antibody disclosed in WO 2005/103081 such as C6, an anti-CD antibody disclosed in WO2003/68821 such as IMMU-106 (from Immunomedics), an anti-CD20 antibody disclosed in WO2004/103404 such as AME-133 (from Applied Molecular Evolution/Lilly), and anti-CD20 antibody disclosed in US 2003/0118592 such as TRU-015 (from Trubion Pharmaceuticals Inc).

HuMax-CD20™ (ofatumumab), described as 2F2 antibody in WO2004/035607, is a fully human IgG1, κ high-affinity antibody targeted at the CD20 molecule in the cell membrane of B-cells. HuMax-CD20™ is in clinical development for the treatment of non-Hodgkin's lymphoma (NHL), chronic lymphocytic leukemia (CLL), and rheumatoid arthritis (RA). See also Teeling et al., Blood, 104, pp 1793 (2004); and Teeling et al., J. Immunology, 177, pp 362-371 (2007).

There is a need for formulating a shear and temperature stable pharmaceutical formulation comprising a protein which is suitable for therapeutic use. Embodiments of such formulations are disclosed herein.

### SUMMARY OF THE INVENTION

The present invention relates to a shear and temperature stable aqueous antibody formulation.

This invention is not to be limited in scope by the specific embodiments described herein. Indeed, various modifications of the invention in addition to those described herein will become apparent to those skilled in the art from the foregoing description. Such modifications are intended to fall within the scope of the appended claims.

Although one embodiment is adapted to a full length monoclonal antibody formulation, it may also be used for the formulation of other classes of antibodies, for example, polyclonal antibodies, or fragments of monoclonal or polyclonal antibodies.

In one embodiment, the invention relates to a protein formulation comprising a therapeutically effective amount of a protein, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0.

In another embodiment, the invention relates to a protein formulation comprising a protein in the concentration range of 20-300 mg/mL, wherein the formulation further comprises 50 mM sodium acetate, 51 mM sodium chloride, 1% arginine free base, 0.05 mM EDTA, 0.02% polysorbate 80, and adjusted to pH 5.5. In another embodiment, the protein is a protein fragment, an antibody, an IgG antibody, a monoclonal antibody, a polyclonal antibody, a monoclonal antibody fragment, a polyclonal fragment, a monoclonal anti-CD20 antibody fragment, a full length anti-CD20 antibody, a monoclonal anti-OSM antibody fragment, a full length anti-OSM antibody, a monoclonal anti-MAG antibody fragment, and a full length anti-MAG antibody. The preferred anti-CD20 antibody is ofatumumab.

In another embodiment, the invention relates to a protein, such as anti-CD20 antibody, formulation comprising a protein, such as an anti-CD20 antibody, in the concentration range of 20-300 mg/mL, wherein the formulation further comprises 50 mM sodium acetate, 51 mM sodium chloride, 1% arginine free base, 0.05 mM EDTA, 0.02% polysorbate 80, and adjusted to pH 5.5. In another embodiment, the protein is another anti-protein, such as, but not limited to, a full length or fragment of an anti-protein antibody, an anti-OSM antibody, an anti-MAG antibody, or an anti-CD20 antibody. The preferred anti-CD20 antibody is ofatumumab.

In yet another embodiment, the invention relates to a protein formulation wherein the formulation is stable for at least 2 years. In another embodiment, the invention relates to a protein formulation wherein the formulation is stable at temperatures up to at least 55° C. In another embodiment, the invention relates to a protein formulation wherein the formulation is stable at a temperature of about 5° C. for at least 2 years. In another embodiment, the invention relates to a protein formulation wherein the formulation is stable at a temperature of about 25° C. for at least 3 months. In another embodiment, the invention relates to a protein formulation wherein the formulation is stable at a temperature of about 40° C. for at least 1 month. In another embodiment, the invention relates to a protein formulation wherein the formulation is stable at a temperature of about 55° C. for at least 1 day. In another embodiment, the invention relates to a protein formulation wherein the formulation is stable at a temperature range of approximately, 5 to 55° C. for at least 1 day with shaking. In another embodiment, the invention relates to a protein formulation wherein the formulation is stable at a temperature range of approximately, 5 to 25° C., 5 to 35° C., 5 to 45° C., 10 to 25° C., 10 to 35° C., 10 to 45° C., 10 to 55° C., 20 to 35° C., 20 to 45° C., or 20 to 55° C. for at least 1 day with shaking.

In another embodiment, the invention relates to a protein formulation wherein the antibody is present in an amount of about 20-300 mg/mL, 50-300 mg/mL, 100-300 mg/mL, 150-300 mg/mL, 200-300 mg/mL, or 250-300 mg/mL.

In another embodiment, the invention relates to a protein formulation wherein sodium acetate is present in an amount of about 50 mM, 40 mM, 45 mM, 55 mM, or 60 mM. In other embodiments, the sodium acetate may be present in an amount of 10 to 100 mM, 20 to 100 mM, 30 to 100 mM, 40 to 100 mM, 50 to 100 mM, 60 to 100 mM, 70 to 100 mM, 25 to 80 mM, or 30 to 70 mM.

In yet another embodiment, the invention relates to a protein formulation wherein acetic acid is present (about 100 mM acetic acid) to adjust the formulation to about pH 5.5. In other embodiments, the pH may be adjusted to pH 5.0, 5.5, 6.0, 6.5 or 7.0. In yet other embodiments of the invention, NaOH or HCl is used to adjust the pH to 5.0, 5.5, 6.0, 6.5 or 7.0.

In yet another embodiment, the invention relates to a protein formulation wherein sodium chloride is present in an amount of about 51 mM, 45 mM, 46 mM, 47 mM, 48 mM, 49 mM, 50 mM, 52 mM, 53 mM, 54 mM, 55 mM. In other embodiments, the sodium chloride may be present in an amount of 25 to 100 mM, 35 to 90 mM, 45 to 80 mM, 25 to 70 mM, or 45 to 70 mM.

In another embodiment, the invention relates to a protein formulation wherein arginine free base is present in an amount of about 1%, 0.7%, 1.3%, or 2.0%. In other embodiments, the arginine free base may be between 0.5 to 5.0%, 0.5 to 2.0%, 0.5 to 2.5%, 0.5 to 3.0%, 0.5 to 3.5%, 0.5 to 4.0%, or 0.5 to 4.5%.

In another embodiment, the invention relates to a protein formulation wherein EDTA is present in an amount of about 0.05 mM, 0.03 mM, 0.04 mM, or 0.06 mM. In other embodiments, the EDTA may be present in an amount of 0.02 mM-0.2 mM, 0.02 mM-0.1 mM, 0.02 mM-0.15 mM, 0.04 mM-0.1 mM, 0.03 mM-0.15 mM, or 0.03 mM-0.2 mM.

In another embodiment, the invention relates to a protein formulation wherein polysorbate 80 is present in an amount of about 0.02%, 0.015%, or 0.025%. In other embodiments, the polysorbate 80 may be present in an amount of 0.01-0.1%, 0.01-0.15%, 0.02-0.2%, 0.02-0.15%, 0.01-0.25%, or 0.01-0.05%.

In another embodiment, the invention relates to a method of treating a disease involving cells expressing a protein by administering to a mammal an anti-protein antibody formulation of the present invention comprising a therapeutically effective amount of an anti-protein antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0. Exemplary “diseases involving cells expressing CD20” that can be treated (e.g., ameliorated) or prevented include, but are not limited to, tumorigenic diseases and immune diseases, e.g., autoimmune diseases. Examples of tumorigenic diseases which can be treated and/or prevented include B cell lymphoma, e.g., NHL, including precursor B cell lymphoblastic leukemia/lymphoma and mature B cell neoplasms, such as B cell chronic lymhocytic leukemia (CLL)/small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL). Examples of immune disorders in which CD20 expressing B cells are involved which can be treated and/or prevented include psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, Omenn's syndrome, chronic renal failure, acute infectious mononucleosis, HIV, and herpes virus associated diseases. Further examples are severe acute respiratory distress syndrome and choreoretinitis. Yet further examples are diseases and disorders caused by infection of B-cells with virus, such as Epstein-Barr virus (EBV). Yet a further example is COPD. Exemplary “diseases involving cells expressing MAG” that can be treated (e.g., ameliorated) or prevented include, but are not limited to the process of neurodegeneration underlying many neurological diseases including acute diseases such as stroke, traumatic brain injury and spinal cord injury as well as chronic diseases including Alzheimer's disease, fronto-temporal dementias (tauopathies), peripheral neuropathy, Parkinson's disease, Huntington's disease and multiple sclerosis. Anti-MAG mabs or MAG antagonists therefore may be useful in the treatment of these diseases, by both ameliorating the cell death associated with these disorders and promoting functional recovery. Exemplary “diseases involving cells expressing OSM” that can be treated (e.g., ameliorated) or prevented include, but are not limited to, inflammatory arthropathies which may be treated according to this invention include rheumatoid arthritis, psoriatic arthritis, juvenile arthritis, inflammatory osteoarthritis and/or reactive arthritis. Inflammatory disorders which may be treated include, amongst others, Crohns disease, ulccerative colitis, gastritis for example gastritis resulting from H. pylori infection, asthma, chronic obstructive pulmonary disease, alzheimer's disease, multiple sclerosis and psoriasis. Anti-OSM mabs or OSM antagonists therefore, for example, may be useful in the treatment of these diseases, by both ameliorating the cell death associated with these disorders and promoting functional recovery.

In yet another embodiment, the invention relates to a method of treating a disease involving cells expressing protein by administering to a mammal an anti-protein antibody formulation of the present invention comprising a therapeutically effective amount of an anti-protein antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0 and wherein the stable antibody formulation is administered orally, parenterally, intranasally, vaginally, rectally, lingually, sublingually, bucally, transdermally, intravenously, or subcutaneously to a mammal.

In yet another embodiment, the invention relates to a method of treating a disease involving cells expressing OSM by administering to a mammal an anti-OSM antibody formulation of the present invention comprising a therapeutically effective amount of an anti-OSM antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0 and wherein the stable antibody formulation is administered orally, parenterally, intranasally, vaginally, rectally, lingually, sublingually, bucally, transdermally, intravenously, or subcutaneously to a mammal.

In yet another embodiment, the invention relates to a method of treating a disease involving cells expressing MAG by administering to a mammal an anti-MAG antibody formulation of the present invention comprising a therapeutically effective amount of an anti-MAG antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0 and wherein the stable antibody formulation is administered orally, parenterally, intranasally, vaginally, rectally, lingually, sublingually, bucally, transdermally, intravenously, or subcutaneously to a mammal.

In yet another embodiment, the invention relates to a method of treating a disease involving cells expressing CD20 by administering to a mammal an anti-CD20 antibody formulation of the present invention comprising a therapeutically effective amount of an anti-CD20 antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2 polysorbate 80 and adjusted to pH 5.0 to 7.0 and wherein the stable antibody formulation is administered orally, parenterally, intranasally, vaginally, rectally, lingually, sublingually, bucally, transdermally, intravenously, or subcutaneously to a mammal.

In yet another embodiment, the invention relates to a method of treating a disease involving cells expressing CD20 by administering to a mammal an anti-CD20 antibody formulation of the present invention comprising an anti-CD20 antibody in the concentration range of 20-300 mg/mL, wherein the formulation further comprises 50 mM sodium acetate, 51 mM sodium chloride, 1% arginine free base, 0.05 mM EDTA, 0.02% polysorbate 80, and adjusted to pH 5.5. The preferred anti-CD20 antibody is ofatumumab.

It is to be understood that both the foregoing summary description and the following detailed description are exemplary and explanatory, and are intended to provide further explanation of the invention as claimed.

The accompanying drawings are included to provide a further understanding of the invention, and are incorporated in, and constitute a part of this specification, illustrate several embodiments of the invention, and together with the description serve to explain the principles of the invention.

### BRIEF DESCRIPTION OF THE FIGURES

FIG. 1 illustrates the standard formulation (RefMat) of anti-CD20 antibody at 20 mg/mL (30 mM citrate, 100 mM NaCl, pH 6.5) in duplicate.

FIG. 2 illustrates one embodiment of the invention (PlatForm) formulation of anti-CD20 antibody at 20 mg/mL (50 mM sodium acetate, sodium chloride (51 mM), 1% arginine free base, 0.05 mM EDTA, 0.02% polysorbate 80, and adjusted to pH to 5.5 with HCl) in duplicate.

FIG. 3 graphically illustrates a comparison of anti-CD20 antibody thermal stability in a formulation embodiment of the invention (PlatForm) and standard formulation buffers (RefMat) by DSC. Thermodynamically, the two formulations are similar as seen by their DSC profiles since the change in apparent Tm is less than 0.5° C. between the formulations.

### DETAILED DESCRIPTION OF THE INVENTION

One embodiment of the present invention relates to shear and temperature stable antibody formulations.

In another embodiment, the invention provides for an unexpected stability seen for a formulation under simultaneous stress conditions of elevated temperature and shaking at 55° C.

A further embodiment of the invention is a more stable formulation than compared to a standard formulation (such as 30 mM citrate, 100 mM NaCl, pH 6.5). The present invention's formulation showed reduced precipitation (remained clear) when subjected to stress conditions but the standard formulation had aggregated. This result was unpredictable because thermodynamically the two formulations are similar as seen by their DSC (differential scanning calorimeter) profiles.

In the description of the present invention, certain terms are used as defined below.

The term “protein formulation” or “antibody formulation” refers to preparations which are in such form as to permit the biological activity of the active ingredients to be unequivocally effective, and which contain no additional components which are toxic to the subjects to which the formulation would be administered.

“Pharmaceutically acceptable” excipients (vehicles, additives) are those which can reasonably be administered to a subject mammal to provide an effective dose of the active ingredient employed. For example, the concentration of the excipient is also relevant for acceptability for injection.

A “stable” formulation is one in which the protein therein essentially retains its physical and/or chemical stability and/or biological activity upon storage. Various analytical techniques for measuring protein stability are available in the art and are reviewed in Peptide and Protein Drug Delivery, 247-301, Vincent Lee Ed., Marcel Dekker, Inc., New York, N.Y., Pubs (1991) and Jones, A. Adv. Drug Delivery Rev. 10: 29-90 (1993), for example. Stability can be measured at a selected temperature for a selected time period. Preferably, the formulation is stable at ambient temperature or at 40° C. for at least 1 month and/or stable at 2-8° C. for at least 1 to 2 years. Furthermore, it is desirable that the formulation be stable following freezing (e.g. to −70° C.) and thawing of the product.

A protein “retains its physical stability” in a biopharmaceutical formulation if it shows little to no change in aggregation, precipitation and/or denaturation as observed by visual examination of color and/or clarity, or as measured by UV light scattering (measures visible aggregates) or size exclusion chromatography (SEC). SEC measures soluble aggregates that are not necessarily a precursor for visible aggregates.

A protein “retains its chemical stability” in a biopharmaceutical formulation, if the chemical stability at a given time is such that the protein is considered to retain its biological activity as defined below. Chemically degraded species may be biologically active and chemically unstable. Chemical stability can be assessed by detecting and quantifying chemically altered forms of the protein. Chemical alteration may involve size modification (e.g. clipping) which can be evaluated using SEC, SDS-PAGE and/or matrix-assisted laser desorption ionization/time-of-flight mass spectrometry (MALDI/TOF MS), for example. Other types of chemical alteration include charge alteration (e.g. occurring as a result of deamidation) which can be evaluated by ion-exchange chromatography, for example.

An antibody “retains its biological activity” in a pharmaceutical formulation, if the change in biological activity of the antibody at a given time is within about 10% (within the errors of the assay) of the biological activity exhibited at the time the pharmaceutical formulation was prepared as determined in an antigen binding assay, for example. Other “biological activity” assays for antibodies are elaborated herein below.

The term “isotonic” means that the formulation of interest has essentially the same osmotic pressure as human blood. In one embodiment, the isotonic formulations of the invention will generally have an osmotic pressure in the range of 250 to 350 mOsm. In other embodiments, isotonic formulations of the invention will have an osmotic pressure from about 350 to 450 mOsm. In yet another embodiment, isotonic formulations of the invention will have an osmotic pressure above 450 mOsm. Isotonicity can be measured using a vapor pressure or ice-freezing type osmometer for example.

As used herein, “buffer” refers to a buffered solution that resists changes in pH by the action of its acid-base conjugate components. In one embodiment, the buffer of this invention has a pH in the range from about 4.5 to about 6.0; in another embodiment, from about 4.8 to about 5.8; and in a further embodiment, a pH of about 5.5. Examples of buffers that will control the pH in this range include acetate (e.g. sodium acetate), succinate (such as sodium succinate), gluconate, histidine, citrate and other organic acid buffers. Where a freeze-thaw stable formation is desired, the buffer is preferably not phosphate.

In a pharmacological sense, in the context of the present invention, a “therapeutically effective amount” of an antibody refers to an amount effective in the prevention or treatment of a disorder for the treatment of which the antibody is effective. A “disorder” is any condition that would benefit from treatment with the antibody. This includes chronic and acute disorders or diseases including those pathological conditions which predispose the mammal to the disorder in question. In a preferred embodiment “disorder” is a disease involving cells expressing CD20.

A “preservative” is a compound which can be included in the formulation to essentially reduce bacterial action therein, thus facilitating the production of a multi-use formulation, for example. Examples of potential preservatives include octadecyldimethylbenzyl ammonium chloride, hexamethonium chloride, benzalkonium chloride (a mixture of alkylbenzyldimethylammonium chlorides in which the alkyl groups are long-chain compounds), and benzelthonium chloride. Other types of preservatives include aromatic alcohols such as phenol, butyl and benzyl alcohol, alkyl parabens such as methyl or propyl paraben, catechol, resorcinol, cyclohexanol, 3-pentanol, and m-cresol. The most preferred preservation herein is benzyl alcohol.

The term “antibody” is used in the broadest sense and specifically covers monoclonal antibodies (including full length monoclonal antibodies), polyclonal antibodies, multispecific antibodies (e.g. bispecific antibodies), and antibody fragments so long as they exhibit the desired biological activity.

“Antibody fragments” comprise a portion of a full length antibody, generally the antigen binding or variable region thereof. Examples of antibody fragments include Fab, Fab′, F(ab′)₂, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determination on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256:495 (1975), or may be made by recombinant DNA methods (see, e.g., U.S. Pat. No. 4,816,567). The “monoclonal antibodies” may also be isolated from phage antibody libraries using the technique described in Clackson et al., Nature 352:624-626 (1991) and Marks et al., J. Mol. Biol. 222:581-597 (1991), for example.

“Humanized” forms of non-human (e.g., murine) antibodies are chimeric antibodies which contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues which are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

“Single-chain Fv” or “sFv” antibody fragments comprise the V_{H} and V_{L} domain of antibody, wherein these domains are present in a single polypeptide chain. Generally, the Fv polypeptide further comprises a polypeptide linker between the V_{H} and V_{L} domains which enables the SFv to form the desired structure for antigen binding. For a view of sFv see Pluckthun in The Pharmacology of Monoclonal Antibodies, vol. 113, Rosenburg and Moore eds. Springer-Verlag, N.Y., pp. 269-315 (1994).

The term “diabodies” refers to small antibody fragments with two antigen-binding sites, which fragments comprise a heavy chain variable domain (V_{H}) connected to a light chain variable domain (V_{L}) in the same polypeptide chain (V_{H} and V_{L}). By using a linker that is too short to allow pairing between the two domains on the same chain, the domains are forced to pair with the complementary domains of another chain and create two antigen-binding sites. Diabodies are described more fully in, for example, EP 404,097; WO 93/11161; and Hollinger et al., Proc. Natl. Acad. Sci. USA 90:6444-6448 (1993).

The expression “linear antibodies” when used throughout the application refers to the antibodies described in Zapata et al. Protein Eng. 8(10):1057-1062 (1995). Briefly, these antibodies comprise a pair of tandem Fd segments (V_{H}—C_{H}—V_{H1}—C_{H1}) which form a pair of antigen binding regions. Linear antibodies can be bispecific or monospecific.

The antibody which is formulated is preferably essentially pure and desirably essentially homogenous (i.e. free from contaminating proteins etc). “Essentially pure” antibody means a composition comprising at least about 90% by weight of the antibody, based on total weight of the composition, preferably at least about 95% by weight. “Essentially homogeneous” antibody means a composition comprising at least about 99% by weight of the antibody, based on total weight of the composition.

“Treatment” refers to both therapeutic treatment and prophylactic or preventative measures. Those in need of treatment include those already with the disorder as well as those in which the disorder is to be prevented.

“Mammal” for purposes of treatment refers to any animal classified as a mammal, including but not limited to humans, domestic and farm animals, and zoo, sports, or pet animals, such as dogs, horses, cats, and cows.

“Stress condition” refers to an environment which is chemically and physically unfavorable for a protein and may render unacceptable protein stability (e.g. thermal, shear, chemical stress).

Size Exclusion Chromatography is a chromatographic method in which particles are separated based on their size or hydrodynamic volume.

Dynamic Light Scattering is a method which measures the time dependence of protein scattered light. Traditionally, this time dependence is processed to yield the hydrodynamic radius of a molecule.

“DSC” refers to differential scanning calorimeter: DSC acquisition parameters: can be but not limited to, 1 mg/ml protein, scan for 5 to 80° C. with a scan rate of 70° C. per hour and 15 minute prewait. A buffer-buffer scan can be acquired first and subtracted from the raw data. The data can be corrected for the buffer and normalized for the protein concentration then plotted. Aggregation can prevent baseline correction.

The following examples are further illustrative of the present invention. The examples are not intended to limit the scope of the present invention, and provide further understanding of the invention.

### EXAMPLES

The invention is further illustrated by way of the following examples which are intended to elucidate the invention. These examples are not intended, nor are they to be construed, as limiting the scope of the invention. Numerous modifications and variations of the present invention are possible in view of the teachings herein and, therefore, are within the scope of the invention. The examples below are carried out using standard techniques, and such standard techniques are well known and routine to those of skill in the art, except where otherwise described in detail.

### Example 1.1

### Preparation of the Platform Formulation Buffer

In one embodiment of the invention, 4 liters of acetate buffer were prepared. In this embodiment, the final buffer was comprised of 50 mM sodium acetate, 0.05 mM EDTA, 51 mM NaCl, 1.0% Arginine, 0.02% Polysorbate 80, pH 5.5. The buffer was prepared by dissolving sodium actetate trihydrate, edetate disodium (EDTA), polysorbate 80 and L-arginine free base into 3.5 L of deoinized water. Once the pH was adjusted to 5.5 using 3N HCl, the volume was brought up to 4.0 L and the buffer was filtered using a 0.45 μm filter unit. The buffer can then be stored at 2-8° C. until use. The formulation “%” described in the present application refers to “% by volume”.

### Example 2.1

### Preparation of Ofatumumab in a Platform Formulation Buffer

In one embodiment of the invention, ofatumumab was diafiltrated into a platform formulation (50 mM Sodium Acetate, 51 mM NaCl, 0.05 mM EDTA, 0.02% Polysorbate 80, and 1.0% Arginine (free-base)) and concentrated for stability. Ofatumumab was diafiltrated in to the platform formulation using a lab-scale tangential flow system with three membranes. After the diafiltration into the platform buffer, ofatumumab was concentrated to a maximum concentration of 179 mg/mL. The entire process took approximately three working days to complete and the yield was 96.1%. Some of the 179 mg/mL was diluted with platform formulation buffer so that a concentration range of ˜20-179 mg/mL could be studied.

### Example 3.1

### Preparation of ofatumumab in Standard and Platform Formulation for General Appearance (GA) Direct Comparison

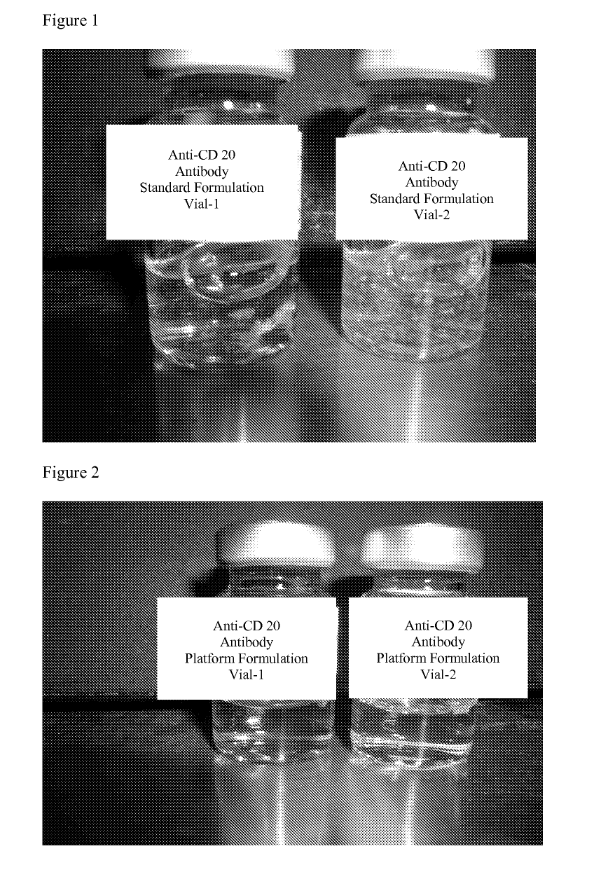

An anti-CD20 antibody (ofatumumab) was prepared in the standard formulation and the platform (one embodiment of the present invention) formulation at a concentration of 20 mg/mL for general appearance in direct comparison over a 12 week time period and for shake experiments. The anti-CD20 antibody in the standard and platform formulations were filtered using a low protein binding 0.2 μm membrane filter. After the filtration, each formulation was filled at 3 mL into 5 cc vials, stoppered and crimped using sterile technique under the clean hood. Two vials of each formulation were placed on a shaker with temperature control. The vials were shaken at 325 RPM at a temperature of 55° C. During the shaking with heat, the general appearance was observed, as described in Example 3.2, periodically over a 42 hour time period. FIGS. 1 and 2 show the standard and platform formulations, respectively, after 18.5 hours of shaking with heat. The overall appearance results of the shake study indicated that the standard formulation will generate particles over time when subjected to shaking at 55 degree C. temperatures more rapidly than the platform formulation.

### Example 3.2

### GA, 18.5 hrs Shake study-General Appearance Ofatumumab, 20 and 100 mg/mL

General appearance (GA) of an anti-CD20 mab shake study samples is presented in the table below. GA was completed using a general method which can be used for an IgG antibody solution which describes color, clarity and visible particulate matter.

| Shake Time Point | Appearance |
|---|---|
| Initial | |
| Standard | Clear, Colorless, 1-2 Particles present |
| Platform | Clear, Colorless, Particle Free |
| 18.5 hours | |
| Standard | Clear, Colorless, Several large Particles Present |
| Platform | Clear, Colorless, Particle Free |
| 42 hours | |
| Standard | Hazy, Colorless, Several Large particles present |
| Platform | Slightly hazy, colorless, particle free |

### Example 4

### To Determine the Thermal Stability of Ofatumumab Solution in the Standard and Platform Buffer by Differential Scanning Calorimetry (DSC)

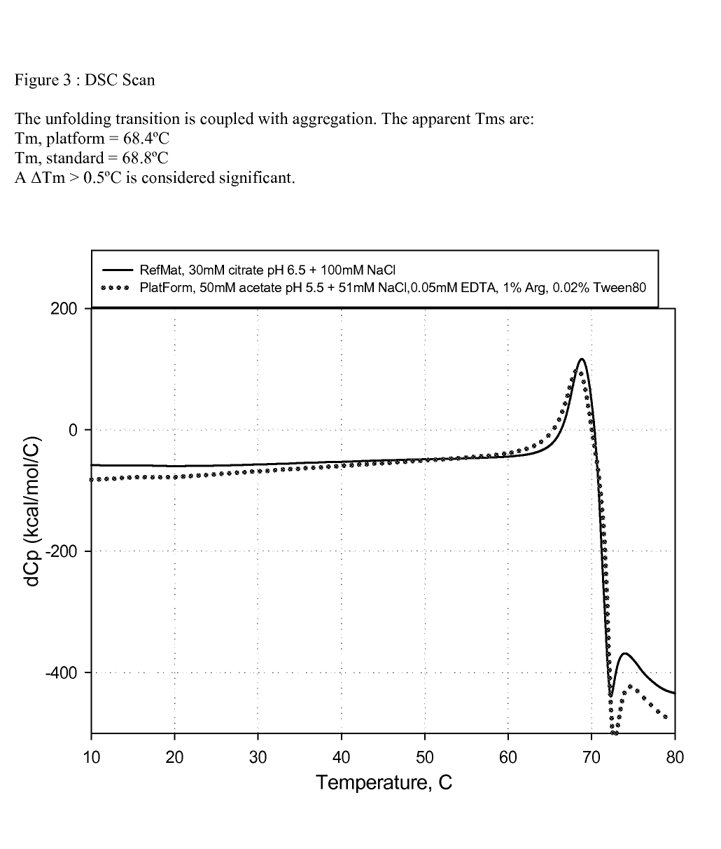

In order to properly complete the testing by DSC, scans of the buffers alone and with protein were acquired. The protein in the standard and platform formulations were diluted to 1 mg/mL as presented in Example 4.1. Data was acquired setting the DSC to scan from 5-80° C. at a scan rate of 70° C. per hour with a 15 minute equilibration before each scan. The volume of the DSC sample cell is ˜0.5 mL. After the scans of the buffer and protein were acquired, the buffer scans could then be subtracted from the protein scan. A concentration of the protein in the samples was obtained to correct for the concentration in each scan (See, Example 4.2). The values for Tᵤₙ, ° C., start of unfolding, Tₘ, ° C., denaturation temperature (at transition maximum) and T_{1/2}, ° C., the width of the peak at half-height (reflect changes in tertiary structure and cooperativity of the transitions) were obtained for ofatumumab for each formulation (See, Example 4.3). The actual DSC scans can be seen in FIG. 3. Based on the results of the DSC, the ofatumumab in either the standard formulation or the platform formulation had similar DSC profiles and therefore would be expected to have similar thermal stability.

### Example 4.1

### Sample Preparation for Biophysical Characterization of Ofatumumab pH Study

### 1. Dilutions

| | | | Dilute to 1 mg/ml | |
|---|---|---|---|---|
| | | Initial | for DSC | |
| | | conc. | ml | ml |
| pH | Buffer | mg/ml | sample | buffer |
| 6.5 | 30 mM citrate, 100 mM NaCl | 17 | 0.1 | 1.6 |
| Standard | | | | |
| Formulation | | | | |
| 5.5 | 50 mM acetate, 51 mM | 20 | 0.075 | 1.43 |
| Platform | NaCl, 0.05 mM EDTA, 1% | | | |
| Formulation | Arg, 0.02% Tween-80 | | | |

### Example 4.2

### A280 Measurements

| | | Initial | Measured conc. of | |
|---|---|---|---|---|
| | | conc. | 0.5 mg/ml dilution | |
| pH | Buffer | mg/ml | mg/ml | mM* |
| 6.5 | 30 mM citrate, 100 mM | 17 | 0.517 | 0.00345 |
| Standard | NaCl | | | |
| Formulation | | | | |
| 5.5 | 50 mM acetate, 51 mM | 20 | 0.444 | 0.00296 |
| Platform | NaCl, 0.05 mM EDTA, | | | |
| Formulation | 1% Arg, 0.02% Tween- | | | |
| | 80 | | | |

*use to normalize DSC scans.

Prep one sample, blank with corresponding buffer, read 3 times. Use 1 cm cuvette. Subtract A320 absorbance before dividing by extinction coefficient (1.49).

### Example 4.3

### DSC Results

| | | | Tᵤₙ, | Tₘ, | T_{1/2}, | |
|---|---|---|---|---|---|---|
| Sample | pH | Buffer | ° C. | ° C. | ° C. | Notes |
| Standard | 6.5 | 30 mM citrate, | 62 | 68.8 | 2.9* | |
| Formulation | | 100 mM NaCl | | | | |
| Platform | 5.5 | 50 mM acetate, | 60 | 68.4 | 3.2* | Similar to |
| Formulation | | 51 mM NaCl, | | | | Standard |
| | | 0.05 mM EDTA, | | | | Formulation |
| | | 1% Arg, | | | | |
| | | 0.02% Tween-80 | | | | |

*The T_{1/2} values were determined manually. The exothermic contribution from aggregation distorts the baseline, thus these values may be artificially small.

### Example 5.1

### Achieving a High Concentration, anti-OSM

In another embodiment, a stirred cell was used that exchanged an anti-OSM antibody gently while stirring above a membrane with a low molecular weight cut-off to not allow loss of protein, a concentration of anti-OSM at 278 mg/mL was achieved in the platform (as described in Example 1.1 above) formulation buffer without NaCl. In addition, using tangential flow (TGF) a concentration of ˜228 mg/mL was achieved in the platform (as described in Example 1.1 above) formulation buffer without NaCl. Finally, material was also prepared using a lab-scale TGF unit with two membranes. Material prepared in the lab-scale unit reached ˜212 mg/mL in the platform (as described in Example 1.1 above) formulation buffer. The material prepared from all three processes was placed on stability and all materials were found to be stable at the storage condition 2-8° C.

### Example 5.1

### Stability Studies, anti-OSM

In one embodiment of the invention, a solution study was created to observe the stability of an anti-OSM antibody at a range of concentrations. The material that was prepared in Example 5.1 via a stirred cell was placed on stability, along with material prepared via the TGF process. Concentrations lower than 212 mg/mL were created by diluting the aOSM at this concentration into formulation buffer. As a result, this study included concentrations that ranged from 95˜278 mg/mL. The 2-8° C. storage condition was accessed as well as several stress conditions including −20, 25 and 40° C. storage. The study lasted 16 weeks. The 16th week samples were also stored at 2-8° C. and tested again at a later time point, 32 weeks. All formulations were prepared in the platform (as described in Example 1.1 above) formulation buffer without the addition of sodium chloride. The sodium chloride is added to the platform formulation to assure an isotonic solution and is not added to assist in stability of the protein. The physical (pH, appearance), biochemical (concentration by A280 nm, SEC-HPLC, cIEF, SDS-PAGE) and activity (binding ELISA) measures indicate that concentrations of an anti-OSM antibody from 95 to 278 mg/mL in the platform formulation and can be maintained at 2-8° C. storage condition for at least 32 weeks. Also, it was identified in this study, aggregation and deamidation can be considered the major degradation pathways for anti-OSM.

In this embodiment of the invention, anti-OSM antibody concentrations of approximately 150 and 200 mg/mL in the platform formulation were subjected to three freeze/thaw cycles and 48 hours of vigorous shaking at 2-8° C. The results at both conditions indicate, that even at the high concentrations, the material was stable after three freeze/thaw cycles and 48 hours of shaking in glass vials by all the physical, biochemical and activity measures employed.

In another embodiment of the invention, lab-scale anti-OSM was prepared at 150 mg/mL in the platform formulation (as described in Example 1.1) and compared to an anti-OSM GMP large-scale batch at 100 mg/mL in the same platform formulation. The anti-OSM antibody was placed at 5, 25, and 40° C., as well as, several frozen conditions including −40 and −70° C. and conditions where the anti-OSM antibody was frozen at −70° C. (flash freezing) and then stored at either −40 or −20° C. The results indicate that anti-OSM antibody at about 150 mg/mL maintains stability at the storage condition of 5° C. and has a similar stability profile as the GMP 100 mg/mL material that was prepared at large-scale by all the physical, chemical and activity measures employed. Both concentrations had similar degradation profiles at the stress thermal conditions studied 25 and 40° C. All the frozen storage conditions appeared to be stable and gave comparable results with the exception of the samples that were frozen at −70° C. and then stored at −20° C. By the 2 week time point, these samples had already begun to show a trend of increased aggregation by SEC-HPLC. This was not seen in the 100 mg/mL sample and appears to be concentration dependent. However, this study suggests that another embodiment of the invention is that a frozen storage condition of −40° C. could be considered if −70° C. storage is unavailable. In yet another embodiment, freezing at −70° C. and subsequent storage at a temperature of −40° C. could also be an alternative.

### Example 6.1

### Pre-formulation and Formulation Studies for Anti-Mag Antibody Used to Justify the Platform Formulation (as Described in Example 1.1)

Thermal Stability: An anti-MAG antibody was used in the following experiment. Ten near isotonic solutions with a pH ranging from 4.0 to 8.5 were prepared. Slide-a-lyser dialysis was employed to produce 10 ml of 10 mg/mL solutions for the experiment. These samples were diluted to 1 mg/mL with relevant buffer for the thermal analysis. The thermal stability of the anti-MAG antibody in solutions with a pH ranging from 4.0 to 8.5 was performed using a Seteram Micro DSC III. Samples were scanned for thermal events from 25° C. to 90° C. at a rate of 0.7° C./min and an isothermal hold before scanning commenced for 30 minutes. Each determination was carried out in duplicate. The reference material was the inert (buffer, all components minus anti-MAG antibody) to resemble each sample and the sample size for reference and sample was identical and close to 0.8 g. All the data suggest that the thermal stability for anti-MAG antibody is good irrespective of pH. Onset of denaturation ranges from 68° C. to 72° C. and precipitation from approximately 75° C. to 85° C. except for solutions with a pH of 4.5 or below in which no or little aggregation or precipitation was observed.

pH Stability Profile: The same material generated for the thermal stability was also used in this experiment. Approximately 1 mL aliquots from each solution were filled into Sarstedt tubes and stored at 50° C. and 5° C. in a temperature controlled cabinet for 1 month. The stability of the samples were compared using a number of biochemical (IEX-HPLC, RP-HPLC, SDS-PAGE and SEC-HPLC) techniques and ELISA as a measure of activity. The IEX-HPLC method failed to produce any results, due to the varying pH in the samples. The results show that all assays agree that the stability of anti-MAG antibody at 50° C. is worst at pH values above 7.0. The ELISA and the RP-HPLC results suggest that the stability of anti-MAG antibody is at an optimum in solutions with a pH ranging from 4.5 to 5.5 the SEC-HPLC results and the non reduced CE-SDS-PAGE results suggest that the optimum stability can be found in the pH range between 5.0 and 6.0.

pH Solubility Profile: The PEG precipitation method was used to determine the solubility of anti-MAG antibody in solutions with pH from 4.0 to 8.5. Sufficient PEG 6000 was added to precipitate 25% to 75% of the protein, ideally 5 but at least 3 values between 25-75% precipitation were obtained. Depending on the pH of the solution between 6.25% and 25% PEG 6000 was added. The mixtures were left overnight, filtered through a 0.2 μm filter and the protein content was determined in the filtrate. A Hewlett Packard 8453 UV detector was used for the analysis of the samples at 280 nm and the concentration of protein was determined using 1.61 E. The log values of the protein concentrations for each solution were plotted against the PEG 6000 concentration used for each precipitation. The intercept on the y-axis indicating the solubility of protein in the test solution. The solubility anti-MAG antibody in solutions with pH from 4.0 to 8.5 determined by the PEG precipitation method and shows that the desired 100 mg/mL cannot be achieved in the pH range between 6.5 and 7.5 without addition of solubilizers. A solubility of 1000 mg/mL is recorded for all results where the log extrapolations gave very high values.

Effect of Shear on Stability: A 2 mL sample of each solution was added to a luminescence cuvette with a stirring flea. The cuvette was placed in a Perkin Elmer LS 50B fluorimeter thermostatted to 20° C. and with stirring on high speed. A measure of the quantity of visible particulates in the stirred cuvette was obtained from luminescence measurements with the excitation and emission wavelength set to 400 nm. Analysis of the stirred sample was carried out every 30 min. The results suggests that anti-MAG antibody is most sensitive to shear stress in solutions with pH values ranging from 6.5 to 5.5 and least sensitive to shear when the pH of the solution is 4.5 or 4.0.

Cu (II) Binding Evaluation: Copper ions have been implemented in degradation of monoclonal antibodies. Any interaction can quickly be visualised spectroscopically. Anti-MAG antibody without Cu(II) added shows no CD signal in the visible range. On addition of up to 90 μM Cu(II) a negative band at 570 nm grew with the amount of Cu(II) added. Precipitate was observed on addition of Cu(II) chloride, on mixing this precipitate disappeared. The titration stopped when the precipitate remained clearly visible following mixing. In order to confirm that the observed changes in scans were not due to the precipitate the 10 mg/mL sample containing 100 μM Cu(II) was filtered and the filtrate rescanned. The scans are sufficiently similar to conclude that the CD signal around 570 nm is real and that interaction between Cu(II) and anti-MAG antibody takes place.

Effect of Buffer Type and Solubilizers on Solubility: Many co-solvents, salts, buffering agents and other excipients affect the solubility/stability of a protein due to differential binding (electrostatic interactions, van der Wales's interactions, hydrogen bonding and other short range forces), which will shift the free energy of protein unfolding. When the free energy for unfolding is increased the protein is stabilized (the solubility increases). When excipients preferentially interacts with the unfolded protein (reduction in free energy for unfolding) the protein is destabilized (the solubility is reduced). The solubility of anti-MAG antibody was compared using acetate or phosphate buffer to achieve the same pH. The solubility was also determined at pH 5.5 and 6.5 and after addition of arginine to the buffer, a known solubilizer for monoclonal antibodies. Purified anti-MAG antibody was dialysed into the test vehicles. The test vehicles were phosphate buffer pH 5.5, acetate buffer pH 5.5+1% arginine, phosphate buffer pH 5.5+1% Arginine, phosphate buffer pH 6.5+1% arginine, all near isotonic. The final concentration of the active was approximately 5 mg/mL. The results indicate that an acetate buffer clearly provides better solubility for anti-MAG antibodies compared with a phosphate buffer. The addition of arginine increased the solubility of anti-MAG antibodies in both types of buffer and at all the tested pH values.

Effect of Chelating Agents and Nitrogen on Stability: Since an interaction between Cu(II) and anti-MAG antibody was identified in a previous experiment (not presented) and EDTA is a good chelator for Cu(II), the addition of EDTA to the vehicle may, therefore reduce the degradation rate. The degradation process itself is fuelled by oxygen, eliminating oxygen from the vehicle and the headspace of the container the material is stored in, may also prevent degradation via this pathway. A heat stability experiment was carried out to confirm the interaction of Cu(II) with anti-MAG antibodies and to evaluate if the addition of EDTA and purging the vehicle and headspace of the container with nitrogen would reduce the degradation of anti-MAG antibodies. Purified Anti-MAG antibody was dialysed into 50 mM acetate buffer pH 5.5 containing NaCl to isotonicity and diluted to 10 mg/mL with the vehicle. This solution was used to make the formulations for the experiment. These samples were incubated at 50° C. for 25 days before analysis. The samples were analysed for stability by SEC-HPLC and ELISA. Both SEC-HPLC and ELISA results suggest that when 0.1 mM and 0.2 mM EDTA had been added to the solution, the stability of AntiMAG was unaffected by the addition of 0.034 mMCu(II) to 0.34 mMCu(II). The addition of 0.34 mMCu(II) to the Anti-MAG solution alone caused extensive degradation of the active. Purging with Nitrogen reduced the degradation of the active compared to the degradation when Cu(II) alone had been added, however replacing oxygen with nitrogen did not appear to reduce the extend of degradation to the same extent as the addition of EDTA had. The SEC-HPLC results also suggested that when neither EDTA nor Nitrogen had been used to slow the degradation and the sample had not been spiked with Cu(II), the appearance of low molecular weight material was slightly higher than in samples with EDTA, this could be due to the presence of small amounts of Cu(II) in the excipients or container closure system.

Effect of Surfactant: Surfactants are amphiphilic molecules, they will, for this reason straddle hydrophobic/hydrophilic interfaces (e.g. air/water or solid/water interfaces). Proteins also adsorb to these types of interfaces, which is a major cause of aggregation and precipitation. Surfactants inhibit interface-induced aggregation by limiting the extent of protein adsorption to hydrophobic/hydrophilic interfaces. As for other excipients surfactants interacts with proteins by differential binding. Many surfactants preferentially bind to the unfolded state, reducing the conformational stability. Studies to determine the lowest concentration of a surfactant to prevent shear-induced aggregation were, therefore undertaken. Purified anti-MAG antibody was initially diluted to 50 mg/mL in a vehicle of acetate buffer pH 5.5 containing 1% arginine. Studies were repeated using 100 mg/mL test solutions and surfactant in the optimum concentration range from the 50 mg/mL study. This was done to conserve active for formulation development. The only surfactant tested was polysorbate 80, since this surfactant has been approved as an excipient for injections and because this surfactant has been used to reduce shear induced aggregation for monoclonal antibodies before. The addition of concentrations of polysorbate 80 from 0.001% to 0.2% was evaluated. The shear stress stability of solutions with polysorbate 80 was compared to the shear stress stability of anti-MAG solutions without polysorbate. The results demonstrate that the addition of polysorbate 80 improved the shear stress stability of anti-MAG and suggest that the addition of 0.02% polysorbate 80 may be sufficient to completely eliminate shear-induced aggregation in the model used. Also, the addition of 0.02% polysorbate 80 almost eliminated shear-induced aggregation in test solutions containing 100 mg/mL of an anti-MAG antibody.

Effect of Buffer Type, Concentration and Sodium Chloride on Stability: Interactions between an excipient and the anti-MAG antibody may effect the long-term stability of the anti-MAG antibody. Such interactions would effect the choice and/or concentration of an excipient. The product could be adjusted to isotonicity by varying the concentration of the buffer, typically however sodium chloride or sucrose is used for this purpose. Active-excipient interactions may aid selection of the most effective way of adjusting the tonicity of a product. Purified anti-MAG antibody was dialysed into the following vehicles: sodium acetate, potassium phosphate and sodium phosphate vehicles at pH 5.5-6.0 with the addition of 1% arginine and sodium chloride to create an isotonic solution. The solutions were then diluted with the appropriate vehicle to 20 mg/mL. One mL aliquots were filled into 2 mL vials. Two vials from each solution were then stored at 5° C. until analysis and 2 vials were stored at 50° C. for 28 days and then analysed. The samples were analysed for stability by SEC-HPLC and ELISA as a measure of activity. The results indicate that varying the concentration of the acetate buffer and adding various concentrations of NaCl had little or no effect on the stability of the active. In addition there is a difference in anti-MAG antibody stability between vehicles containing acetate and phosphate buffer. This difference could however be due to a difference in pH rather than the buffer. The ELISA assay did not suggest any difference in anti-MAG antibody stability in the different vehicles. The study suggested that the concentration of a buffer had no effect on the stability of the active and that the osmolality could be adjusted with NaCl without effecting the stability. The study also indicated that the acetate buffer might provide better long-term stability compared to a phosphate buffer.

Effect of Light: The stability of proteins is effected by exposure to light to a varying degree depending on the presence of certain amino acids, in particular tryptophan on the outer surface of the macromolecule. For peptides identifying tryptophan in the molecule can indicate the sensitivity to light for that molecule. For large proteins identifying tryptophan is not sufficient to assess the photosensitivity of the molecule, the position of the amino acid in the tertiary structure also need to be known. To ensure the reliability of pre-formulation/formulation studies of macromolecules, their sensitivity to light must be eliminated from the studies. For this reason the sensitivity of anti-MAG antibody to light was determined. The stability of anti-MAG antibody in light will also be determined according to ICH guidance during GMP stability studies. Purified anti-MAG antibody was used for the experiment. The concentration of the anti-MAG antibody was 100 mg/mL and the vehicle used was 50 mM acetate buffer, pH 5.5 containing 0.05 mM EDTA and 0.02% Polysorbate 80. One mL aliquots were filled into 2 mL type I glass vials and closed with West stoppers. One vial was stored at 5° C. until analysis. Four were placed in an Atlas Suntest CPS cabinet. One of these vials had been wrapped to exclude light. The cabinet was set to give an exposure of 300 Watt-hours/m₂. One vial was removed from the cabinet following 2 hours, 4 hours and 6 hours exposure. The wrapped vial was removed after 6 hours in the cabinet. The samples were analysed by SEC-HPLC. The results show a slight increase in the amount of aggregates on exposure to light. The increase can be considered small compared with the exposure of light. Based on this light study, anti-MAG antibody was, for the purposes of the development studies, not considered sensitive to light.

Osmolality: Pharmaceutically the need for isotonicity of injections is governed by the route of administration. Solutions for subcutaneous injection need not necessarily be made isotonic, although isotonicity reduces pain on injection. Solutions for intravenous injection should generally be isotonic. Hypotonic solutions may cause haemolysis of red blood cells and hypertonic solutions may damage the walls of the veins. Anti-MAG antibody may be given by IV injection. The osmolality of serum is 305 mOsm. The adverse effects from IV injection of hypotonic solutions is considered more serious than injection of slightly hypertonic solutions, the target osmolality for the anti-MAG antibody injection was, therefore set to 315 mOsm with a range of 280 mOsm to 350 mOsm. The osmolality of solutions of the individual excipients and the active was determined. The omsolality of the formulation was then determined and the formulation was adjusted with NaCl until the osmolality was 315 mOsm for the complete formulation. The contribution from the individual components in the formulation except NaCl was calculated to be 185 mOsm. To achieve an osmolality of 315 mOsm 3.9 mg NaCl first added. Experimentally, the resulting osmolality of this formulation was 304 mOsm, for this reason an additional amount of NaCl was added, making the total amount of NaCl in the formulation 4.2 mg/mL.

### Platform Formulation

From the development studies the platform formulation given in Table 1 is proposed.

TABLE 1
Platform Formulation for anti-MAG antibody (IgG antibody)
100 mg in 1 mL

| Ingredient | Quantity per unit | |
|---|---|---|
| anti-MAG antibody | 100.00 | mg |
| Sodium Acetate trihydrate, Ph. Eur/USP (50 mM) | 5.94 | mg |
| Disodium Edetate dihydrate Ph. Eur/USP | 0.0186 | mg |
| (0.05 mM) | | |
| Polysorbate 80 Ph. Eur/USP Veg. Org. (0.02%) | 0.20 | mg |
| Arginine hydrochloride Ph. Eur/USP (1.0%) | 10.00 | mg |
| Sodium Chloride Ph Eur/USP (71.9 mM) | 4.20 | mg |
| Acetic Acid, Glacial (0.38 mg) Ph. Eur/USP | q.s. to pH 5.5 | |
| Water for injections Ph. Eur/USP | To 1.0 | mL |
| Nitrogen Ph Eur/USP | 0.75 | atm |

In more detailed embodiments, the anti-CD20 antibody formulation of the present invention can be used to treat a subject with a tumorigenic disorder, e.g., a disorder characterized by the presence of tumor cells expressing CD20 including, for example, B cell lymphoma, e.g., NHL. Examples of tumorigenic diseases which can be treated and/or prevented include B cell lymphoma, e.g., NHL, including precursor B cell lymphoblastic leukemia/lymphoma and mature B cell neoplasms, such as B cell chronic lymhocytic leukemia (CLL)/small lymphocytic lymphoma (SLL), B cell prolymphocytic leukemia, lymphoplasmacytic lymphoma, mantle cell lymphoma (MCL), follicular lymphoma (FL), including low-grade, intermediate-grade and high-grade FL, cutaneous follicle center lymphoma, marginal zone B cell lymphoma (MALT type, nodal and splenic type), hairy cell leukemia, diffuse large B cell lymphoma, Burkitt's lymphoma, plasmacytoma, plasma cell myeloma, post-transplant lymphoproliferative disorder, Waldenstrom's macroglobulinemia, and anaplastic large-cell lymphoma (ALCL).

Further examples of B cell non-Hodgkin's lymphomas are lymphomatoid granulomatosis, primary effusion lymphoma, intravascular large B cell lymphoma, mediastinal large B cell lymphoma, heavy chain diseases (including .gamma., .mu., and .alpha. disease), lymphomas induced by therapy with immunosuppressive agents, such as cyclosporine-induced lymphoma, and methotrexate-induced lymphoma.

In a further embodiment, anti-CD20 antibody formulation of the present invention can be used to treat Hodgkin's lymphoma.

Examples of immune disorders (diseases) in which cells expressing CD20 which can be treated and/or prevented by an anti-CD20 antibody formulation of the present invention include autoimmune disorders, such as psoriasis, psoriatic arthritis, dermatitis, systemic scleroderma and sclerosis, inflammatory bowel disease (IBD), Crohn's disease, ulcerative colitis, respiratory distress syndrome, meningitis, encephalitis, uveitis, glomerulonephritis, eczema, asthma, atherosclerosis, leukocyte adhesion deficiency, multiple sclerosis, Raynaud's syndrome, Sjogren's syndrome, juvenile onset diabetes, Reiter's disease, Behcet's disease, immune complex nephritis, IgA nephropathy, IgM polyneuropathies, immune-mediated thrombocytopenias, such as acute idiopathic thrombocytopenic purpura and chronic idiopathic thrombocytopenic purpura, hemolytic anemia, myasthenia gravis, lupus nephritis, systemic lupus erythematosus, rheumatoid arthritis (RA), atopic dermatitis, pemphigus, Graves' disease, Hashimoto's thyroiditis, Wegener's granulomatosis, Omenn's syndrome, chronic renal failure, acute infectious mononucleosis, HIV, and herpes virus associated diseases. Further examples are severe acute respiratory distress syndrome and choreoretinitis. Furthermore, other diseases and disorders include those caused by or mediated by infection of B-cells with virus, such as Epstein-Barr virus (EBV).

Further examples of inflammatory, immune and/or autoimmune disorders in which autoantibodies and/or excessive B lymphocyte activity are prominent and which can be treated and/or prevented by anti-CD20 antibody formulation of the present invention, include the following:

vasculitides and other vessel disorders, such as microscopic polyangiitis, Churg-Strauss syndrome, and other ANCA-associated vasculitides, polyarteritis nodosa, essential cryoglobulinaemic vasculitis, cutaneous leukocytoclastic angiitis, Kawasaki disease, Takayasu arteritis, giant cell arthritis, Henoch-Schonlein purpura, primary or isolated cerebral angiitis, erythema nodosum, thrombangiitis obliterans, thrombotic thrombocytopenic purpura (including hemolytic uremic syndrome), and secondary vasculitides, including cutaneous leukocytoclastic vasculitis (e.g., secondary to hepatitis B, hepatitis C, Waldenstrom's macroglobulinemia, B-cell neoplasias, rheumatoid arthritis, Sjogren's syndrome, or systemic lupus erythematosus); further examples are erythema nodosum, allergic vasculitis, panniculitis, Weber-Christian disease, purpura hyperglobulinaemica, and Buerger's disease; skin disorders, such as contact dermatitis, linear IgA dermatosis, vitiligo, pyoderma gangrenosum, epidermolysis bullosa acquisita, pemphigus vulgaris (including cicatricial pemphigoid and bullous pemphigoid), alopecia greata (including alopecia universalis and alopecia totalis), dermatitis herpetiformis, erythema multiforme, and chronic autoimmune urticaria (including angioneurotic edema and urticarial vasculitis); immune-mediated cytopenias, such as autoimmune neutropenia, and pure red cell aplasia; connective tissue disorders, such as CNS lupus, discoid lupus erythematosus, CREST syndrome, mixed connective tissue disease, polymyositis/dermatomyositis, inclusion body myositis, secondary amyloidosis, cryoglobulinemia type I and type II, fibromyalgia, phospholipid antibody syndrome, secondary hemophilia, relapsing polychondritis, sarcoidosis, stiff man syndrome, and rheumatic fever; a further example is eosinophil fasciitis; arthritides, such as ankylosing spondylitis, juvenile chronic arthritis, adult Still's disease, and SAPHO syndrome; further examples are sacroileitis, reactive arthritis, Still's disease, and gout; hematologic disorders, such as aplastic anemia, primary hemolytic anemia (including cold agglutinin syndrome), hemolytic anemia secondary to CLL or systemic lupus erythematosus; POEMS syndrome, pernicious anemia, and Waldemstrom's purpura hyperglobulinaemica; further examples are agranulocytosis, autoimmune neutropenia, Franklin's disease, Seligmann's disease, .mu.-chain disease, paraneoplastic syndrome secondary to thymoma and lymphomas, and factor VIII inhibitor formation; endocrinopathies, such as polyendocrinopathy, and Addison's disease; further examples are autoimmune hypoglycemia, autoimmune hypothyroidism, autoimmune insulin syndrome, de Quervain's thyroiditis, and insulin receptor antibody-mediated insulin resistance; hepato-gastrointestinal disorders, such as celiac disease, Whipple's disease, primary biliary cirrhosis, chronic active hepatitis, and primary sclerosing cholangiitis; a further example is autoimmune gastritis; nephropathies, such as rapid progressive glomerulonephritis, post-streptococcal nephritis, Goodpasture's syndrome, membranous glomerulonephritis, and cryoglobulinemic nephritis; a further example is minimal change disease; neurological disorders, such as autoimmune neuropathies, mononeuritis multiplex, Lambert-Eaton's myasthenic syndrome, Sydenham's chorea, tabes dorsalis, and Guillain-Barr's syndrome; further examples are myelopathy/tropical spastic paraparesis, myasthenia gravis, acute inflammatory demyelinating polyneuropathy, and chronic inflammatory demyelinating polyneuropathy; cardiac and pulmonary disorders, such as chronic obstructive pulmonary disease (COPD), fibrosing alveolitis, bronchiolitis obliterans, allergic aspergillosis, cystic fibrosis, Loffler's syndrome, myocarditis, and pericarditis; further examples are hypersensitivity pneumonitis, and paraneoplastic syndrome secondary to lung cancer; allergic disorders, such as bronchial asthma and hyper-IgE syndrome; a further example is amaurosis fugax; opthalmologic disorders, such as idiopathic chorioretinitis; infectious diseases, such as parvovirus B infection (including hands-and-socks syndrome); and gynecological-obstretical disorders, such as recurrent abortion, recurrent fetal loss, and intrauterine growth retardation; a further example is paraneoplastic syndrome secondary to gynaecological neoplasms; male reproductive disorders, such as paraneoplastic syndrome secondary to testicular neoplasms; and transplantation-derived disorders, such as allograft and xenograft rejection, and graft-versus-host disease.

In one embodiment, the disease involving cells expressing CD20 is an inflammatory, immune and/or autoimmune disorder selected from ulcerative colitis, Crohn's disease, juvenile onset diabetes, multiple sclerosis, immune-mediated thrombocytopenias, such as acute idiopathic thrombocytopenic purpura and chronic idiopathic thrombocytopenic purpura, hemolytic anemia (including autoimmune hemolytic anemia), myasthenia gravis, systemic sclerosis, and pemphigus vulgaris.

In another embodiment, the process of neurodegeneration underlies many neurological diseases including acute diseases such as stroke, traumatic brain injury and spinal cord injury as well as chronic diseases including Alzheimer's disease, fronto-temporal dementias (tauopathies), peripheral neuropathy, Parkinson's disease, Huntington's disease and multiple sclerosis. Anti-MAG mabs or MAG antagonists therefore may be useful in the treatment of these diseases, by both ameliorating the cell death associated with these disorders and promoting functional recovery.

In another embodiment, inflammatory arthropathies which may be treated according to this invention include rheumatoid arthritis, psoriatic arthritis, juvenile arthritis, inflammatory osteoarthritis and/or reactive arthritis. Inflammatory disorders which may be treated include, amongst others, Crohns disease, ulccerative colitis, gastritis for example gastritis resulting from H. pylori infection, asthma, chronic obstructive pulmonary disease, alzheimer's disease, multiple sclerosis and psoriasis. Anti-OSM mabs or OSM antagonists therefore, for example, may be useful in the treatment of these diseases, by both ameliorating the cell death associated with these disorders and promoting functional recovery.

This invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A protein formulation comprising a therapeutically effective amount of a protein, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0.

2. The protein formulation of claim 1, wherein the protein is selected from the group consisting of: a protein fragment, an antibody, an IgG antibody, a monoclonal antibody, a polyclonal antibody, a monoclonal antibody fragment, and a polyclonal fragment.

3. The protein formulation of claim 1, wherein the protein is a monoclonal antibody.

4. The protein formulation of claim 1, wherein the formulation is stable at a temperature of about 5° C. for at least 2 years.

5. The protein formulation of claim 1, wherein the formulation is stable at a temperature of about 25° C. for at least 3 months.

6. The protein formulation of claim 1, wherein the formulation is stable at a temperature of about 40° C. for at least 1 month.

7. The protein formulation of claim 1, wherein the formulation is stable at a temperature of about 55° C. for at least 1 day.

8. The protein formulation of claim 1, wherein the formulation is stable at a temperature range of approximately, 5 to 55° C. for at least 1 day with shaking.

9. The protein formulation of claim 1, wherein the formulation is present in an amount of about 20-300 mg/mL.

10. The protein formulation of claim 1, wherein the sodium acetate is present in an amount of about 50 mM.

11. The protein formulation of claim 1, wherein the anti-CD20 antibody formulation is about pH 5.5.

12. The protein formulation of claim 1, wherein the sodium chloride is present in an amount of about 51 mM.

13. The protein formulation of claim 1, wherein the arginine free base is present in an amount of about 1%.

14. The protein formulation of claim 1, wherein the EDTA is present in an amount of about 0.05 mM.

15. The protein formulation of claim 1, wherein the polysorbate 80 is present in an amount of about 0.02%.

16. A protein formulation comprising a protein in the concentration range of 20-300 mg/mL, wherein the formulation further comprises 50 mM sodium acetate, 51 mM sodium chloride, 1% arginine free base, 0.05 mM EDTA, 0.02% polysorbate 80, and adjusted to pH 5.5.

17. The protein formulation of claim 16, wherein the protein is selected from the group consisting of an anti-OSM antibody, an anti-MAG antibody, and an anti-CD20 antibody.

18. (canceled)

19. (canceled)

20. A method of treating a disease involving cells expressing a protein in a mammal, comprising administering an anti-protein antibody formulation comprising a therapeutically effective amount of an anti-protein antibody, wherein the formulation further comprises 10 to 100 mM sodium acetate, 25 to 100 mM sodium chloride, 0.5 to 5% arginine free base, 0.02 to 0.2 mM EDTA, 0.01 to 0.2% polysorbate 80 and adjusted to pH 5.0 to 7.0.

21. The method of claim 20, wherein the protein is selected from the group consisting of MAG and OSM.

22. (canceled)

23. A method of treating a disease involving cells expressing a protein in a mammal, comprising administering an anti-protein antibody formulation comprising an anti-protein antibody in the concentration range of 20-300 mg/mL, wherein the formulation further comprises 50 mM sodium acetate, 51 mM sodium chloride, 1% arginine free base, 0.05 mM EDTA, 0.02% polysorbate 80, and adjusted to pH 5.5.

24. The method of claim 23, wherein the protein is selected from the group consisting of MAG and OSM.

25. (canceled)

26. The method according to claim 20, wherein the formulation is administered to a mammal by intravenous or subcutaneous route.

27. The method according to claim 23, wherein the formulation is administered to a mammal by intravenous or subcutaneous route.
